(19) United States
(12) Patent Application Publication
(10) Pub. No.: US 2011/0160809 A1
(43) Pub. Date: Jun. 30, 2011

(54) PULSE CHARGE LIMITER

(76) Inventor: Timothy J. Cox, Leonard, TX (US)

(21) Appl. No.: 12/982,136

(22) Filed: Dec. 30, 2010

Related U.S. Application Data
(60) Provisional application No. 61/290,944, filed on Dec. 30, 2009.

(51) Int. Cl. A61N 1/08; A61N 1/36; H02J 1/00

(52) U.S. Cl. 607/63; 323/351

(57) ABSTRACT

There is disclosed a device for limiting the amount of electrical charge delivered from an implantable pulse generator to an electrode of an implantable neurostimulation system. The device, connectable between the pulse generator and an electrode, includes a capacitor connected between two depletion mode n-channel MOSFETs with the gate terminals of each of the MOSFETs being connected to opposite terminals of the capacitor, and the source terminals of the MOSFETs being connected to the same terminal of the capacitor as the gate terminal of the other MOSFET. A switch can also be connected in parallel to the capacitor to facilitate the draining of the stored energy stored in the capacitor. Additionally, circuitry can be connected between the two MOSFETs, with the circuitry configured to resonate at a know frequency of electromagnetic interference.

### CROSS-REFERENCE TO RELATED APPLICATIONS

This application claims the benefit of U.S. Provisional Application No. 61/290,944, filed Dec. 30, 2009, which is incorporated herein by reference.

### TECHNICAL FIELD

The present application is generally related to a device a method for limiting the amount of current delivered to an electrode in an electrical stimulation system for patient therapy.

### BACKGROUND

Neurostimulation systems are devices that generate electrical pulses and deliver the pulses to nerve tissue to treat a variety of disorders. Spinal cord stimulation (SCS) is an example of neurostimulation in which electrical pulses are delivered to nerve tissue in the spine for the purpose of chronic pain control. Other examples include deep brain stimulation, cortical stimulation, cochlear nerve stimulation, peripheral nerve stimulation, vagal nerve stimulation, sacral nerve stimulation, etc. While a precise understanding of the interaction between the applied electrical energy and the nervous tissue is not fully appreciated, it is known that application of an electrical field to spinal nervous tissue can effectively mask certain types of pain transmitted from regions of the body associated with the stimulated nerve tissue. Specifically, applying electrical energy to the spinal cord associated with regions of the body afflicted with chronic pain can induce “paresthesia” (a subjective sensation of numbness or tingling) in the afflicted bodily regions. Thereby, paresthesia can effectively mask the transmission of non-acute pain sensations to the brain.

Neurostimnulation systems generally include a pulse generator and one or several leads, The pulse generator is typically implemented using a metallic housing that encloses circuitry for generating the electrical pulses. The pulse generator is usually implanted within a subcutaneous pocket created under the skin by a physician. The leads are used to conduct the electrical pulses from the implant site of the pulse generator to the targeted nerve tissue. The leads typically include a lead body of an insulative polymer material with embedded wire conductors extending through the lead body. Electrodes on a distal end of the lead body are coupled to the conductors to deliver the electrical pulses to the nerve tissue.

There are concerns related to delivering electrical charge to electrodes in multi-channel neurostimulators systems which utilize multiple pathways to deliver electrical charge to the target area or target tissue of the patient. At least one of the concerns is related limiting the maximum amount of current density at a particular electrode to prevent or reduce damage to tissue at the electrode. As a result of the multiple pathways, it is very difficult for the IPG to predict which path will have the highest electrical charge delivery. If the charge density occurring at any one of the electrodes becomes too high, the tissue in proximity to the corresponding electrode may be damaged.

Additionally, there are concerns related to the compatibility of neurostimulation systems with magnetic resonance imaging (MRI). MRI generates cross-sectional images of the human body by using nuclear magnetic resonance (NMR). The MRI process begins with positioning the patient in a strong, uniform magnetic field. The uniform magnetic field polarizes the nuclear magnetic moments of atomic nuclei by forcing their spins into one of two possible orientations. Then, an appropriately polarized pulsed RF field, applied at a resonant frequency, forces spin transitions between the two orientations. Energy is imparted into the nuclei during the spin transitions. The imparted energy is radiated from the nuclei as the nuclei “relax” to their previous magnetic state. The radiated energy is received by a receiving coil and processed to determine the characteristics of the tissue from which the radiated energy originated to generate the intra-body images.

Existing neurostimulation systems are designated as being contraindicated for MRI, because the time-varying magnetic RF field causes the induction of current which, in turn, can cause significant heating of patient tissue due to the presence of metal in various system components. The induced current can be “eddy current” and/or current caused by the “antenna effect.” As used herein, the phrase “MRI-induced current” refers to eddy current and/or current caused by the antenna effect.

“Eddy current” refers to current caused by the change in magnetic flux due to the time-varying RF magnetic field across an area bounding conductive material (i.e., patient tissue). The time-varying magnetic RF field induces current within the tissue of a patient that flows in closed-paths. When a pulse generator and an implantable lead are placed within tissue in which eddy currents are present, the implantable lead and the pulse generator provide a low impedance path for the flow of current. Electrodes of the lead provide conductive surfaces that are adjacent to current paths within the tissue of the patient. The electrodes are coupled to the pulse generator through a wire conductor within the implantable lead. The metallic housing (the “can”) of the pulse generator provides a conductive surface in the tissue in which eddy currents are present. Thus, current can flow from the tissue through the electrodes and out the metallic housing of the pulse generator. Because of the low impedance path and the relatively small surface area of each electrode, the current density in the patient tissue adjacent to the electrodes can be relatively high. Accordingly, resistive heating of the tissue adjacent to the electrodes can be high and can cause significant, irreversible tissue damage.

Also, the “antenna effect” can cause current to be induced which can result in undesired heating of tissue. Specifically, depending upon the length of the stimulation lead and its orientation relative to the time-varying magnetic RF field, the wire conductors of the stimulation lead can each function as an antenna and a resonant standing wave can be developed in each wire. A relatively large potential difference can result from the standing wave thereby causing relatively high current density and, hence, heating of tissue adjacent to the electrodes of the stimulation lead.

### SUMMARY

Disclosed herein is a device for limiting the amount of electrical charge being delivered from an implantable pulse generator to an electrode of an implantable neurostimulation system. The device, connected between the pulse generator and an electrode, includes a capacitor connected between two depletion mode n-channel MOSFETs with the gate terminals of each of the MOSFETs being connected to opposite terminals of the capacitor, and the source terminals of the MOSFETs being connected to the same terminal of the capacitor as the gate terminal of the other MOSFET. A switch can also be connected in parallel to the capacitor to facilitate the draining of the stored energy stored in the capacitor. Additionally, circuitry can be connected between the two MOSFETs, with the circuitry configured to resonate at a know frequency of electromagnetic interference.

The foregoing has outlined rather broadly certain features and/or technical advantages in order that the detailed description that follows may be better understood. Additional features and/or advantages will be described hereinafter which form the subject of the claims. It should be appreciated by those skilled in the art that the conception and specific embodiment disclosed may be readily utilized as a basis for modifying or designing other structures for carrying out the same purposes. It should also be realized by those skilled in the art that such equivalent constructions do not depart from the spirit and scope of the appended claims. The novel features, both as to organization and method of operation, together with further objects and advantages will be better understood from the following description when considered in connection with the accompanying figures. It is to be expressly understood, however, that each of the figures is provided for the purpose of illustration and description only and is not intended as a definition of the limits of the appended claims.

### BRIEF DESCRIPTION OF THE DRAWINGS

FIG. 1 illustrates a stimulation system according to a representative embodiment.

FIG. 2 is a schematic of an embodiment of the present invention.

FIG. 3 is a schematic of another embodiment of the present invention.

FIG. 4 is a schematic of yet another embodiment of the present invention.

### DETAILED DESCRIPTION

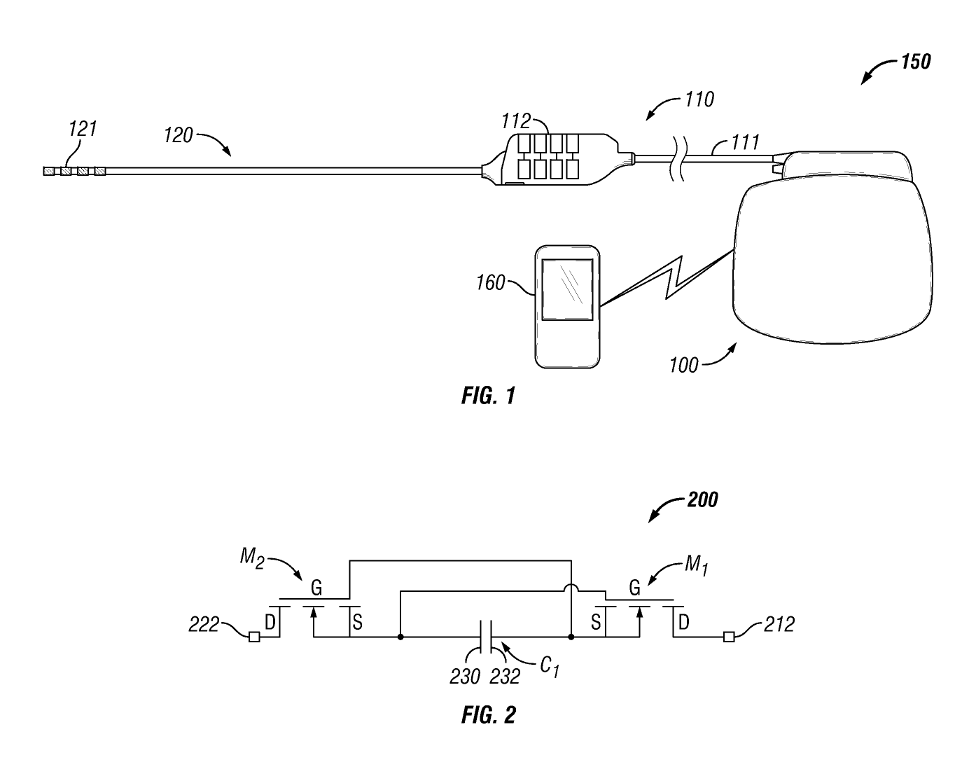

Referring now to FIGS. 1-4, there are illustrated embodiments of the present invention, wherein like elements are illustrated with the same reference numerals and letters throughout the various figures.

FIG. 1 depicts stimulation system 150 that generates electrical pulses for application to tissue of a patient according to one representative embodiment. According to one preferred embodiment, system 150 is a deep brain stimulation system. In other embodiments, system 150 may stimulate any other tissue in a patient such as cortical brain tissue, spinal cord tissue, peripheral nerve tissue, cardiac tissue, etc.

System 150 includes implantable pulse generator 100 that is adapted to generate electrical pulses for application to tissue of a patient. Implantable pulse generator 100 typically comprises a metallic housing that encloses pulse generating circuitry, control circuitry, communication circuitry, battery, charging circuitry, etc. of the device. The control circuitry typically includes a microcontroller or other suitable processor for controlling the various other components of the device. An example of pulse generating circuitry is described in U.S. Patent Publication No. 20060170486 entitled “PULSE GENERATOR HAVING AN EFFICIENT FRACTIONAL VOLTAGE CONVERTER AND METHOD OF USE,” which is incorporated herein by reference. A processor and associated charge control circuitry for an implantable pulse generator is described in U.S. Patent Publication No. 20060259098, entitled “SYSTEMS AND METHODS FOR USE IN PULSE GENERATION,” which is incorporated herein by reference. Circuitry for recharging a rechargeable battery of an implantable pulse generator using inductive coupling and external charging circuits are described in U.S. patent Ser. No. 11/109,114, entitled “IMPLANTABLE DEVICE AND SYSTEM FOR WIRELESS COMMUNICATION,” which is incorporated herein by reference. An example of a DBS implantable pulse generator is the LIBRA® pulse generator available from St. Jude Medical Neuromodulation Division (Plano, Tex.). Examples of commercially available implantable pulse generators for spinal cord stimulation are the EON® and EON® MINI pulse generators available from St. Jude Medical Neuromodulation Division.

Stimulation system 150 further comprises stimulation lead 120. Stimulation lead 120 comprises a lead body of insulative material about a plurality of conductors that extend from a proximal end of lead 120 to its distal end. The conductors electrically couple a plurality of electrodes 121 to a plurality of terminals (not shown) of lead 120. The terminals are adapted to receive electrical pulses and the electrodes 121 are adapted to apply stimulation pulses to tissue of the patient. Also, sensing of physiological signals may occur through electrodes 121, the conductors, and the terminals. Additionally or alternatively, various sensors (not shown) may be located near the distal end of stimulation lead 120 and electrically coupled to terminals through conductors within the lead body 111.

Stimulation system 150 further comprises extension lead 110. Extension lead 110 is adapted to connect between pulse generator 100 and stimulation lead 120. That is, electrical pulses are generated by pulse generator 100 and provided to extension lead 110 via a plurality of terminals (not shown) on the proximal end of extension lead 110. The electrical pulses are conducted through conductors within lead body 111 to housing 112. Housing 112 includes a plurality of electrical connectors (e.g., “Bal-Seal” connectors) that are adapted to connect to the terminals of lead 120. Thereby, the pulses originating from pulse generator 100 and conducted through the conductors of lead body 111 are provided to stimulation lead 120. The pulses are then conducted through the conductors of lead 120 and applied to tissue of a patient via electrodes 121.

Although, lead 120 and lead extension 110 are adapted to support four independent electrodes 121, any suitable number of electrodes can be supported on a respective lead.

In practice, stimulation lead 120 is implanted within a suitable location within a patient adjacent to tissue of a patient to treat the patient's particular disorder(s). For example, in deep brain stimulation for Parkinson's disease, electrodes 121 may be implanted within or immediately adjacent to the subthalamic nucleus. The lead body extends away from the implant site and is, eventually, tunneled underneath the skin to a secondary location. Housing 112 of extension lead 110 is coupled to the terminals of lead 120 at the secondary location and is implanted at that secondary location. Lead body 111 of extension lead 110 is tunneled to a third location for connection with pulse generator 100 (which is implanted at the third location).

Controller 160 is a device that permits the operations of pulse generator 100 to be controlled by a clinician or a patient after pulse generator 100 is implanted within a patient. Controller 160 can be implemented by utilizing a suitable handheld processor-based system that possesses wireless communication capabilities. The wireless communication functionality can be integrated within the handheld device package or provided as a separate attachable device. The interface functionality of controller 160 is implemented using suitable software code for interacting with the clinician and using the wireless communication capabilities to conduct communications with IPG 100.

Controller 160 preferably provides one or more user interfaces that are adapted to allow a clinician to efficiently define one or more stimulation programs to treat the patient's disorder(s). Each stimulation program may include one or more sets of stimulation parameters including pulse amplitude, pulse width, pulse frequency, etc. IPG 100 modifies its internal parameters in response to the control signals from controller 160 to vary the stimulation characteristics of stimulation pulses transmitted through stimulation lead 120 to the tissue of the patient.

Referring now to FIG. 2, there is illustrated an embodiment of a pulse charge limiting device or circuit 200 which limits the amount of electrical charge being delivered from the IPG 100 to one of the electrodes 121. Circuit 200 includes two depletion mode n-channel MOSFETs M1 and M2 and a capacitor C1. As illustrated, the gate terminal of M1 and the source terminal of M2 are both connected to the same terminal of capacitor C1, and the gate terminal of M2 and the source terminal of M1 are both connected to the opposite terminal of capacitor C1. Circuit 200 is to be connected in system 150, intermediate IPG 100 and electrodes 121, and electrically connecting the drain 212 of M1 to the pulse generating circuitry of IPG 100 and by electrically connecting the drain 222 of M2 to one of the electrodes 121. Although it is contemplated that circuit 200 could be connected intermediate IPG 100 and electrodes 121 at a location based upon a user's preference, good results have further been achieved by locating circuit 200 within the housing of IPG 100.

As illustrated in FIG. 2, MOSFETs M1 and M2 are three terminal devices with terminals designated gate (G), drain (D) and source (S). in each of M1 and M2, the channel resistance between drain and source is controlled by a voltage between the gate and source (V_{gs}) such that if the channel resistance is designated R_{ds}, then R_{ds} is proportional to the square of Vₚ plus V_{gs}, where Vₚ is a threshold potential difference. A similar device may have R_{ds} proportional to the exponential function of Vₚ plus V_{gs}. Thus there is a finite and small resistance between drain and source terminals when there is no voltage across the gate-source terminals. Therefore, the current drawn into the gate terminals by M1 and M2, during any range of V_{gs}, is less than the current drawn through or flowing in the capacitor 230.

In an initial state, the current in and voltage across capacitor 230 are initially zero. Then, a gradually increasing potential difference is applied across the drain terminals of M1 and M2. When the potential at the drain of M1 is at a lower potential than at the drain of M2, the current will flow into M2 and out of M1, and a charge builds up on plate 230 of capacitor C1 and is diminished on plate 232 of capacitor C1.

Because equal and opposite charge on a capacitor is proportional to voltage then a potential difference integrates (mathematically) the flow of charge (current) into one “plate” of the capacitor, and an equal current flows out of the other “plate”. That voltage is also applied to the gate and source terminals of M1 and M2 being of a positive polarity V_{gs} for M1 and a negative polarity V_{gs} for M2. The negative polarity on M2 causes it's channel resistance to increase, while that on M1 decreases, because of the positive polarity of V_{gs} so applied. As the magnitude of V_{gs} on M1 reaches Vₚ the channel resistance of M1 rapidly increases: M1 is then said to be “off” even though a finite but high resistance exists. The current flowing through C1 is then rapidly impeded, the voltage across C1 ceases to increase, and a negligible amount of current flows through the output terminals, (the drain terminals of M1 and M2). The steady voltage across capacitor C1 is practically equal to the constant Vₚ of M1. The final charge on the plates of C1 is given by: Q=C1×Vₚ. This is equivalent to the charge that flowed into the external circuit as V_{gs} was increasing. Thus the electrical charge being delivered through circuit 200 is limited to C1Vₚ.

In operation, the channel resistance of each MOSFET M1 and M2 is low while the potential difference across the capacitor C1 is zero. When a current is forced to flow through M1 and M2 and the capacitor C1 by the pulse generating circuitry of IPG 100, the voltage across capacitor C1 increases in proportion to the amount of the charge passed. At a voltage equal to the threshold of voltage of one of M1 and M2, the source-to-gate potential difference is enough to cause the channel resistance of the MOSFET to increase exponentially. This causes the potential difference across the capacitor C1 and one pair of drain-to-source terminals to rapidly reach a predetermined limit voltage of the generating current source. At or near the limit voltage, the generated current substantially decreases, so much so that the pulse current will cease, and no more charge will be delivered from the IPG 100 to the connected electrode of electrodes 121.

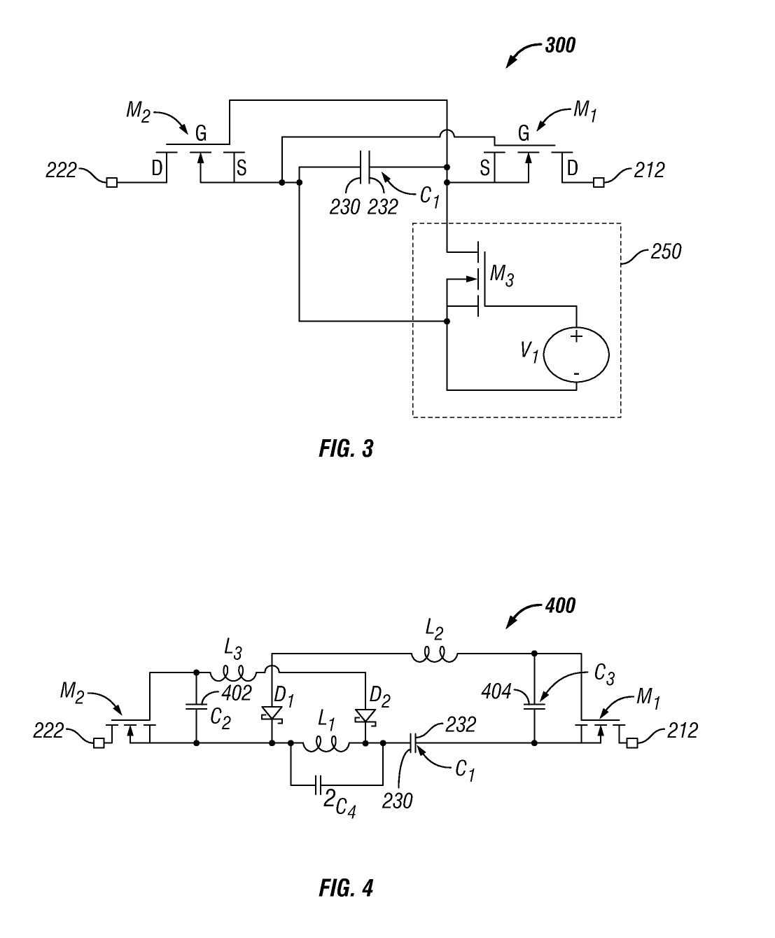

Referring now to FIG. 3, there is illustrated another embodiment of a pulse charge limiting device or circuit 300 as similarly shown and described above with reference to circuit 200 of FIG. 2, and further includes a switch 250 connected in parallel with capacitor C1. Switch 250 is used to drain or remove the charge accumulated in C1 in order to reset circuit 200 back to an initial or preset set state, with the switch being closed during the reset procedure. As illustrated switch 250 includes a MOSFET M3 and a voltage source V1 which operate to control switch 250, thereby facilitating the removal of charge from capacitor C1. Although shown with a MOSFET and voltage source, it is contemplated that switch 250 could have varying designs in order to facilitate the removal of charge from capacitor C1, such as but not limited to using JFET in place of the MOSFET, or utilizing a current source with a bipolar junction transistor.

Referring now to FIG. 4, there is illustrated another embodiment of a pulse charge limiting device or circuit 400 as similarly shown and described herein above, and further utilizing additional circuitry designed to resonate at a known frequency of electromagnetic interference, such as 64 MHz RF emitted from a 1.5 Telsa MRI machine. As illustrated, in addition to MOSFETs M1 and M2, and capacitor C1 circuit 200 includes capacitors C2, C3 and C4; inductors L1, L2 and L3; and diodes D1 and D2.

Inductor L1 is connected across or in parallel with the capacitor C1 located between source terminals of M1 and M2. The resulting parallel tuned, or “tank”, circuit is designed to resonate at a known frequency of electromagnetic interference. The parallel resonance function causes the alternating voltage across inductor L1 and capacitor C4 to be larger than that of a simple capacitor of the same impedance. Specifically, the impedance at resonance is given by:

L1/(C4R1)_when_frequency=½π\sqrt{L1C4}

where R1 is the internal resistance of the inductor L1.

A relatively small current will cause a relatively large voltage to be applied to both diodes D1 and D2, with D1 being in series with inductor L2 and MOSFET M1, and D2 being in series with inductor L1 and MOSFET M2, Both diodes D1 and D2 conduct when peak alternating potential differences across them reach nominal threshold voltages, such as by way of example, 0.1V for backward (modified Esaki or tunnel) diodes, 0.3V for germanium and Schottky diodes and 0.6V for silicon diodes. As a result of conduction, the series tuned circuits, comprising inductor L3 and capacitor C2, and inductor L2 and capacitor C3, receive small bursts of current on each cyclic peak of alternating voltage across the parallel tuned circuit. The Q-multiplication function of inductor L3 with capacitor C2, and inductor L2 with capacitor C3, also tuned at or close to the same known frequency of electromagnetic interference, causes a large voltage to build up across capacitor C2 and capacitor C3. This large voltage is an effect of “pumping” charge through the corresponding diodes, which accumulates as a negative charge in plate 402 of capacitor C2 and plate 404 of capacitor C3, in turn creating large negative voltage V_{gs} across the MOSFETs M1 and M2. Both MOSFETs, M1 and M2, therefore turn “off” in response to low levels of interfering frequency current, thereby preventing the delivery of electrical charge to the connected electrode. In addition to a high channel resistance both MOSFETs exhibit a low capacitance from drain to source terminals, so that reactance at the known frequency, exhibited at the drain terminals, is much larger than channel resistance of the MOSFETs M1 and M2 in their “on” state. If the reactance is large enough, it is contemplated that circuit 400 could be made with a single one of the pairs of series and parallel tuned circuits. For example, if the reactance is large enough in MOSFET M2, inductor L2 and diode D1 could be replaced by short circuits, and capacitor C3 removed. In that case, MOSFET M1 responds only to relatively low frequency voltages across capacitor C1 due to current flowing from left to right in circuit 400.

Although certain representative embodiments and advantages have been described in detail, it should be understood that various changes, substitutions and alterations can be made herein without departing from the spirit and scope of the appended claims. Moreover, the scope of the present application is not intended to be limited to the particular embodiments of the process, machine, manufacture, composition of matter, means, methods and steps described in the specification. As one of ordinary skill in the art will readily appreciate when reading the present application, other processes, machines, manufacture, compositions of matter, means, methods, or steps, presently existing or later to be developed that perform substantially the same function or achieve substantially the same result as the described embodiments may be utilized. Accordingly, the appended claims are intended to include within their scope such processes, machines, manufacture, compositions of matter, means, methods, or steps.

What is claimed is:

1. A pulse charge limiter, comprising:
at least a first and a second MOSFET; and
a capacitor connected intermediate the first MOSFET and the second MOSFET.

2. The pulse charge limiter of claim 1, wherein the first MOSFET and the second MOSFET each include a gate, a drain and a source, and wherein the capacitor includes a first terminal and a second terminal, and further wherein the gate of the second MOSEFT is connected to the first terminal of the capacitor.

3. The pulse charge limiter of claim 2, wherein the gate of the second MOSFET is further connected to the source of the first MOSFET.

4. The pulse charge limiter of claim 3, wherein the gate of the first MOSFET is connected to the second terminal of the capacitor.

5. The pulse charge limiter of claim 4, wherein the gate of the first MOSFET is further connected to the source of the second MOSFET.

6. The pulse charge limiter of claim 5, and further including a switch connected in parallel to the capacitor.

7. The pulse charge limiter of claim 5, wherein said switch includes a MOSFET.

8. The pulse charge limiter of claim 2, and further including tuning circuitry connected between the first MOSFET and the second MOSFET.

9. A device for limiting the amount of electrical charge delivered to an electrode from a pulse generator, the device comprising:
a first MOSFET and a second MOSFET, and
a capacitor connected intermediate the first MOSFET and the second MOSFET.

10. The device of claim 9, wherein the first MOSFET and the second MOSFET each include a gate, a drain and a source, and wherein the capacitor includes a first terminal and a second terminal, and further wherein the gate of the second MOSEFT is connected to the first terminal of the capacitor.

11. The device of claim 10, wherein the gate of the second MOSFET is further connected to the source of the first MOSFET.

12. The device of claim 11, wherein the gate of the first MOSFET is connected to the second terminal of the capacitor.

13. The device of claim 12, wherein the gate of the first MOSFET is further connected to the source of the second MOSFET.

14. The device of claim 13, wherein at least one of the first MOSFET and the second MOSFET is a depletion mode n-channel MOSFET.

15. The device of claim 13, and further including a switch connected in parallel to the capacitor.

16. The device of claim 15, wherein said switch includes a MOSFET and a voltage source.

17. The pulse charge limiter of claim 2, and further including tuning circuitry connected between the first MOSFET and the second MOSFET.

18. A device for use in a in a neurostimulation system for limiting the amount of magnetically induced current delivered via a lead to an electrode, the device comprising:
a first MOSFET and a second MOSFET;
a capacitor connected intermediate the first MOSFET and the second MOSFET; and
circuitry configured to resonate at a selected frequency of electromagnetic interference.

19. The device as recited in claim 18, wherein the circuitry includes a first inductor connected to the first capacitor and to the source of the second MOSFET, and further includes a first diode connected to a second inductor, with the first diode connected to the source of the second MOSFET and the inductor connected to the gate of the first MOSFET, and further including a second capacitor connected between the gate and the source of the second MOSFET and a third capacitor connected between the gate and source of the first MOSFET.

20. The device as recited in claim 19, wherein the circuitry further includes a second diode connected to a third inductor, with the second diode connected between the first inductor and the first capacitor and the third inductor connected to the gate of the second MOSFET.
